(19) United States
(12) Patent Application Publication
(10) Pub. No.: US 2016/0310553 A1
(43) Pub. Date: Oct. 27, 2016

(54) FLAX EXTRACTS, PROCESSES, COMPOSITIONS AND METHODS AND USES THEREOF

(71) Applicant: UNIVERSITY OF SASKATCHEWAN, Saskatchewan (CA)

(72) Inventors: Andrew A. Olkowski, Saskatoon (CA); Bernard Laarveld, Saskatoon (CA)

(73) Assignee: University of Saskatchewan, Saskatoon (CA)

(21) Appl. No.: 15/104,631

(22) PCT Filed: Dec. 19, 2014
(86) PCT No.: PCT/CA2014/000900

§ 371 Date: Jun. 15, 2016

Related U.S. Application Data
(60) Provisional application No. 61/918,463, filed on Dec. 19, 2013.

(51) Int. Cl. A61K 36/55; A23K 50/75; A23L 33/105; A23K 50/70; A61K 9/00; A23K 10/30

(52) U.S. Cl. CPC A61K 36/55; A61K 9/0095; A23K 10/30; A23L 33/105; A23K 50/70; A23K 50/75; A61K 2236/13; A61K 2236/331; A61K 2236/53; A23V 2002/00

(57) ABSTRACT

The disclosure provides a process of obtaining a simplified flax extract by water extraction followed by mechanical extraction. The disclosure further provides compositions comprising the flax extract produced by the process and methods and uses thereof for treating parasitic infections and inflammatory conditions.

### RELATED APPLICATIONS

The present application claims the benefit of priority of U.S. provisional patent application No. 61/918,463 filed on Dec. 19, 2013, the contents of which are incorporated herein by reference in their entirety.

### FIELD

The present disclosure relates to simplified flax extracts, a process for preparing the extracts and compositions, methods and uses thereof.

### BACKGROUND

Flax (Linum usitatissimum) has a long history as a food and fibre crop. In the early 1990's flaxseed was rediscovered for its nutritional qualities and its potential content of natural health products. Over the last two decades, more intensive research has led to the identification and characterization of bioactive compounds such as lipids, lignans, and soluble fibre. However, the health benefits of flax are still largely overlooked by contemporary medicine.

Flaxseed is probably best known for its unique lipid quality and composition. Approximately 35% of flaxseed mass consists of oil. Flax-derived oil is a very rich source of omega 3 fatty acids, especially α-linolenic acid (a particular form of omega-3 fatty acid), and for this reason consumption of flax oil is attributed to several health benefits.

Flaxseed oil also contains natural hydrophobic cyclic peptides (cyclolinopeptides/CLPs) comprising eight or nine amino acid residues. The health benefits of products derived from flax cyclic peptides include prevention of conditions such as enteritis, skin problems, and bone degeneration. In addition cyclolinopeptides show anti-parasitic, anti-fungal, and anti-microbial activity (for details see patent application WO 2013/091071).

In WO2013/091071, the anti-coccidial properties of flax cyclic peptides were investigated in vitro using ruminant specific Eimeria oocysts (isolated from clinical cases of coccidiosis diagnosed in sheep, goat, and calves), and avian Eimeria isolated from chickens.

Coccidiosis is a parasitic disease that is a significant health problem in all animals, and especially in the intensive animal industry. According to industry experts, in the near future, development of new strategies for control of coccidiosis will be required. In food producing animals, control of coccidiosis is costly. For instance, the estimated global economic impact of coccidiosis control in poultry exceeds $3 billion (Peek and Landman, 2011).

There is a dearth of recent estimates of costs associated with coccidiosis in livestock, but based on a review of recent livestock industry and extension sources, it is apparent that coccidiosis in ruminants (sheep, goat, cattle) is a major problem. Of note, coccidiosis has been listed as one of the five most economically important diseases in poultry and livestock industries.

Infection of animals with coccidia appears to be very common throughout the world. The disease may exhibit three levels of severity: (1) coccidiasis, a mild infection, causing no observable effects, (2) subclinical coccidiosis, resulting in slight but economically important reductions of growth and feed utilization; and (3) clinical coccidiosis with high morbidity and mortality rates. Coccidiosis usually results in high morbidity and mortality. However, it should be noted that clinically affected animals that survive an outbreak of coccidiosis remain “poor doers” and take significantly longer to mature and breed. Inasmuch as mortality causes a direct loss, subclinical cases present hidden economic costs because the disease usually goes undetected for a long time. Subclinical coccidiosis has been described as a “stealthy intruder”, because it robs producers' gain and performance dollars without any obvious clues of the ongoing problem.

Historically, the strategy of coccidiosis treatment and control has been based on administration of anti-coccidial drugs. However, over time coccidia have developed resistance to drugs, and resistance to drugs is now recognized as a major cause of the failure to control coccidiosis.

Therefore, novel approaches to control coccidiosis in farm animals are urgently needed. However, there is a notable reluctance of the pharmaceutical industry to invest in the development of anticoccidial drugs, as the food animal industry is facing considerable pressure to abolish use of these antibiotic type drugs. In this context, using natural-products provides a highly desirable alternative means of disease control.

Avian trichomoniasis is a parasitic disease that may affect many bird species including domestic fowl, but it is most prevalent among domestic pigeons and wild doves. The causative agent is a flagellated protozoan organism Trichomonas gallinae that invades mouth, esophagus, crop, and other organs. In infected birds, the organism multiplies rapidly, and the clinical course of the disease can be very aggressive.

The first lesions appear as small, yellowish foci on the oral mucosa, but rapidly coalesce to form extensive masses that may completely fill the oral cavity, and block the esophagus. In acute form of the disease death may occur suddenly. In chronic cases, infected pigeons may initially show diminished appetite, but eventually will stop feeding. Ruffled and dull plumage feather is a characteristic sign. As the disease advances the birds lose weight, and become weak and listless.

The severity of the disease depends on the susceptibility of the bird and on the pathogenic potential of the strain of the parasite. Adult birds may recover from the infection and become resistant to reinfection, but still carry the parasite. The infection is readily transmitted from parent to offspring in the normal feeding process. In young birds the infection is rapid, and if left untreated almost always fatal. Treatments include metronidazole and dimetridazole administered in the drinking water.

Capillaria is a genus of nematodes in the family Capillariidae. Approximately 300 species of this genus have been described. Capillaria spp. exemplify a large family of parasitic roundworms that infect livestock, poultry, dogs, cats, and many species of wild mammals, birds, and fish. Some species are also human parasites. Capillaria worms are omnipresent throughout the world. In endemic regions 100% of wild animals may be infected with some Capillaria species.

In generality of cases, the infection spreads by ingestion of embryonated eggs by a suitable host. Adult worms invade the host and lay hundreds of eggs. The eggs in the environment develop to infective stage. The development period varies depending on species. For example, pigeon capillaria oocysts need just a few days to develop outside the pigeon. Infective eggs hatch in the intestine, releasing larvae. The larvae penetrate into the intestinal wall and reach the portal vein system to be carried to the predilection sites, where they complete development to adult worms of both sexes and reproduce.

Some Capillaria species can be highly pathogenic, causing severe disease. Once established these intestinal worms can cause severe symptoms such as diarrhea, weakness, weight loss and in laying hens a drop in egg production. Control of Capillaria infections can be challenging as these parasites often demonstrate multiple drug resistance.

Capillaria infections are very common in pigeons. Adult worms are microscopic in size and burrow in the lining of the intestine, gizzard, ventriculus, and even the crop. Morbidity and mortality are very high in young birds, but even adult birds can die from starvation due to these worms.

Consumption of flax oil has been also been associated with anti-inflammatory effects and other flax phytochemicals have also shown high potential as immune and inflammation modulators. For instance, flax cyclolinopeptides have been shown to exhibit immunosuppressive activity (Wieczorek et al., 1991; Morita et al., 1997; Gorski et al., 2001; Matsumoto et al., 2001).

Inflammation is a normal defense mechanism that protects the host from infection and adverse effects of other insults causing tissue injury. In the situation of bacterial or fungal invasion, inflammatory response is associated with innate defenses which initiate pathogen killing. However, in any instances of insults (microbial, mechanical, chemical, or toxic) causing tissue injury, inflammatory responses are essential in tissue repair processes and help to restore homeostasis at infected or damaged sites.

Typically, an inflammatory response is characterized by hyperemia, swelling, elevated temperature, pain, and impairment or loss of local physiological/biochemical tissue or organ function. This process involves interactions amongst many cellular elements, and further involves the production of numerous chemical mediators.

Normal physiological inflammatory responses are well regulated, self-limiting, and resolve rapidly without causing any excessive damage to the host. When controlled properly, inflammatory responses are essential in maintaining tissue health and homeostasis.

Pathological inflammation develops when the host tolerance and/or processes regulating inflammatory responses are impaired or lost, and this will inevitably lead to severe damage of the host tissues. Irrespective of the cause, the pathological inflammatory responses involve several major events culminating in massive release of mediators from leukocytes at the site of inflammation. These may include lipid mediators such as prostaglandins and leukotrienes, peptide mediators such as cytokines, reactive oxygen species, amino acid derivatives (e.g., histamine), and enzymes such as matrix proteases.

These factors normally play a major role in host defense and tissue repair, but when produced inappropriately or in an unregulated fashion they cause damage to host tissues, leading to disease. Several of these mediators may act to amplify the inflammatory process acting, for example, as chemoattractants. Some of the inflammatory mediators may escape the inflammatory site into the circulation and from there they can exert systemic effects. For example, the cytokine interleukin (IL)-6 induces hepatic synthesis of the acute phase protein C-reactive protein, while the cytokine tumour necrosis factor elicits metabolic effects within skeletal muscle, adipose tissue and bone.

The inflammatory process contributes to a range of acute and chronic conditions characterized by the production of inflammatory cytokines, arachidonic acid−derived eicosanoids such as prostaglandins, thromboxanes, leukotrienes, and oxidized derivatives (for review see Calder, 2010). Consumption of flaxseed oil is believed to increase concentration of α-linolenic acid in blood plasma and decrease the production of arachidonic acid- and eicosanoid-derived inflammatory mediators (Huwiler and Pfeilschifter, 2009; Russo, 2009).

The immunosuppressive effects reported for flaxseed oil cyclolinopeptides (CLPs) have been attributed to their inhibition of T-cell-mediated immunity. For example, CLP A inhibits T-cell proliferation in response to concanavalin A. As a result of this discovery and the known role of T-cells in immunity (especially related to graft rejection and T-cell action), the following observations have also been reported in the literature: CLPs mitigate delayed hypersensitivity, skin allograft rejection and graft vs. host reactions. CLPs inhibit interleukin-alpha and interleukin-2 and temper post-adjuvant polyarthritis and haemolytic immunity (For review see Siemion et al., 1999, Picur et al., 2006).

### SUMMARY

The present inventors developed methods that provide biologically active flax phytochemicals using simplified flax extracts.

Using a stepwise extraction procedure of flaxseeds, a simplified flax extract was obtained. One aspect of the simplified flax extract was the apparent ability of the flax extract to control the development of Eimeria oocysts. Further experiments in vitro confirmed this anticoccidial effect, where both coccidiocidal and coccidiostatic activity were observed.

The present inventors have shown that there is no need to extract and purify biologically active compounds from flax in order to achieve a desirable pharmacological effect. This can be achieved using proper selective sequences of extraction procedures.

In vivo studies demonstrated several novel pharmacological effects that are associated with the simplified flax extract, including anti-parasitic activity and anti-inflammatory activity. In particular, in vivo studies using the avian model demonstrated the following effects of treatment with flax extract: anti-parasitic activity, decreased risk of inflammatory bowel disease, decreased risk of necrotic enteritis, enhanced healing and regeneration of intestinal tissue affected by enteritis, anti-inflammatory activity, and prevention of foot pad necrosis.

Accordingly, the present disclosure provides a process of obtaining a flax extract comprising subjecting seeds to a water extraction process followed by a mechanical extraction process and purifying the liquid therefrom.

In one embodiment, the process comprises:

a) washing flax seeds;

b) subjecting the washed flax seeds to the water extraction process;

c) removing the water soluble fraction;

d) subjecting the extracted seeds of c) to the mechanical extraction process; and

e) purifying the liquid from d).

In an embodiment, the flax seeds are washed in water at a temperature of 20 to 70 degrees Celsius. In another embodiment, the seeds are washed at a ratio of 1:10 to 1:2 seeds:water. The washed seeds are optionally strained and rinsed under running water. In an optional embodiment, the washing is repeated.

In an embodiment, the water extraction is carried out at a temperature of 20 to 120° C., optionally, at about 110-120° C. The extraction is optionally carried out at a ratio of 1:100 to 1:4 seeds to water. The water extraction process step may be repeated as required to remove water soluble compounds. The water-extracted seeds are optionally strained and rinsed under running water.

In an embodiment, the water extraction process is repeated at least once prior to purifying the liquid in e).

In another embodiment, the mechanical extraction process comprises mechanical shear force mixing, homogenization, microfluidization, physical grinding, or ultrasonic-assisted extraction or a combination thereof. The mechanical extraction process is optionally performed at a ratio of 1:100 to 1:20 seeds to water.

In yet another embodiment, the seeds are washed after the water extraction process and before the mechanical extraction process, optionally under running water.

In another embodiment, the ratio of seeds to water during the mechanical extraction process is between about 1:100 and about 1:20 w/v.

In a further embodiment, the liquid is purified by subjecting the extracted seeds to centrifugation, gravity sedimentation and/or filtration after the mechanical extraction process and collecting the liquid therefrom.

In a particular embodiment, the process comprises:

a) washing flax seeds;

b) subjecting the washed flax seeds to a subcritical water extraction process;

c) removing the water soluble fraction;

d) subjecting the extracted seeds of c) to mechanical shear force grinding;

e) subjecting the ground seeds of d) to the ultrasonic-assisted extraction; and

f) purifying the liquid from e).

In another aspect, the present disclosure provides a flax extract produced by the processes disclosed herein.

In an embodiment, the flax extract has anti-parasitic and/or anti-inflammatory activity.

In yet another aspect, the present disclosure provides a feed or food stuff, pharmaceutical composition or a food supplement composition, comprising the flax extract produced by the process disclosed herein. In an embodiment, the composition is in the form of a liquid, paste, gel or solid.

In yet another aspect, the present disclosure provides methods and uses of the flax extract, feed or food stuff, pharmaceutical composition or food supplement for treatment.

In one embodiment, there is provided a method of treating a parasitic infection comprising administering the flax extract, feed or food stuff, pharmaceutical composition or a food supplement composition disclosed herein to an animal in need thereof. Also provided herein is use of the flax extract, feed or food stuff, pharmaceutical composition or a food supplement composition disclosed herein for treating a parasitic infection in an animal in need thereof. Further provided is the flax extract, feed or food stuff, pharmaceutical composition or a food supplement composition disclosed herein for use in treating a parasitic infection in an animal in need thereof. Even further provided is a flax extract produced by a process disclosed herein for the manufacture of a medicament for treating a parasitic infection in an animal in need thereof.

In one embodiment, the parasitic infection is a coccidial infection. In another embodiment, the parasitic infection is a trichomoniasis infection. In yet another embodiment, the parasitic infection is a Capillaria infection.

In another embodiment, there is provided a method of treating an inflammatory condition, disease or disorder comprising administering the flax extract, feed or food stuff, pharmaceutical composition or a food supplement composition disclosed herein to an animal in need thereof. Also provided herein is use of the flax extract, feed or food stuff, pharmaceutical composition or a food supplement composition disclosed herein for treating an inflammatory condition, disease or disorder in an animal in need thereof. Further provided is the flax extract, feed or food stuff, pharmaceutical composition or a food supplement composition disclosed herein for use in treating an inflammatory condition, disease or disorder in an animal in need thereof. Even further provided is a flax extract produced by a process disclosed herein for the manufacture of a medicament for treating an inflammatory condition, disease or disorder in an animal in need thereof.

In an embodiment, the inflammatory condition, disease or disorder affects the integument, mucosal membranes, intestine or stomach of the animal.

In another embodiment, the inflammatory condition, disease or disorder is an inflammatory bowel lesion, necrotic enteritis, gizzard ulceration, skin lesion (e.g. foot pad necrosis).

In an embodiment, the animal is an agricultural animal, a zoo animal, a domestic animal, an aquatic species or a companion animal. In one embodiment, the animal is a bird. In another embodiment, the animal is a human. In yet another embodiment, the animal is a ruminant. In yet a further embodiment, the aquatic species is a fish or shellfish, such as a shrimp.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 is a micrograph of the simplified flaxseed extract. The vesicles size (diameter) in this particular product was found to be in the range of low micron and sub-micron, with the majority being in lower nanometer range. Image was digitally enlarged to cumulative magnification approximately 1000× from an original magnification of 400×.

FIG. 2 depicts the effect of flaxseed extract treatment on Eimeria sporulation. Representative of oocysts from control (a), and treatment (b). Notably, oocysts in control (a) showed vigorous sporulation which is evidenced by the characteristic presence of four spores in every oocyst (arrow). In contrast, sporulation was completely halted by treatment with flax extract, which is evidenced by the absence of characteristic spores in oocysts (arrow).

FIGS. 3a and 3b show representative examples of differences in inflammatory bowel lesions observed in the intestines of a normal population of broilers (poultry grown for meat production) offered tap drinking water (a, arrows) and broilers offered drinking water amended with flax extract (b, arrows). Two sets of specimens shown in bottom part of FIG. 4b without arrows represent relatively normal intestinal mucosa. Notably, the inflammatory responses in the intestinal mucosa are more pronounced with respect to both magnitude and severity in control broilers (FIG. 4a, arrows) in comparison to broilers drinking water amended with flax extract (FIG. 4b, arrows).

FIGS. 4a and 4b show representative examples of differences in necrotic lesions observed in the intestines of a population of broilers at increased risk of morbidity and mortality offered tap drinking water (a, arrows) and offered drinking water amended with flax extract (b, arrows). The acronyms on the respective specimens refer to anatomical section of the intestines describing duo=duodenum; jej=jejunum; ile=ileum; p=proximal; m=medial; d=distal.

FIG. 5 shows differences in feet lesions observed in broilers from the control group (a) and broilers offered drinking water amended with flax extract (b).

FIG. 6 shows Eimeria oocyst counts in fecal droppings of broilers infected with sporulated Eimeria oocysts offered tap drinking water (control) and broilers offered drinking water amended with flax extract (treated).

FIG. 7 shows representative examples of differences in lesions observed in the gizzards of broilers offered tap drinking water (top panel, arrows) and broilers offered drinking water amended with flax extract (bottom panel, arrows).

FIG. 8 shows representative examples of developing Capillaria oocysts morphology isolated from control birds (FIG. 5a, b and c) and from birds drinking water amended with flax extract (FIG. 5d, e and f).

### DETAILED DESCRIPTION

As used herein “a”, “an” and/or “the” includes one and/or more than one.

As used herein the term “about” means than the value or range of values can be greater than or lesser than the stated value or range of values by 10%, but is not intended to limit any value or range of values to only this broader definition. Each value or range of values preceded by the term “about” is also intended to encompass the stated absolute value or range of values.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. The term “treatment or treating” in some embodiments means preventing disease at a subclinical stage.

The term a “therapeutically effective amount”, “effective amount” or a “sufficient amount” of a composition of the present disclosure is a quantity sufficient to, when administered to the subject, including a mammal, for example a human, effect beneficial or desired results, including clinical results, and, as such, an “effective amount” or synonym thereto depends upon the context in which it is being applied. In the context of disease, therapeutically effective amounts of the agents are used to treat, modulate, attenuate, reverse, or affect disease, condition or disorder, such as the parasitic or inflammatory condition. An “effective amount” is intended to mean that amount of a composition that is sufficient to treat, prevent or inhibit such disorders, conditions or diseases. The amount of a given agent that will correspond to such an amount will vary depending upon various factors, such as the given agent, the pharmaceutical formulation, the route of administration, the type of condition, disease or disorder, the identity of the subject or host being treated, and the like, but can nevertheless be routinely determined by one skilled in the art. As defined herein, a therapeutically effective amount of an agent may be readily determined by one of ordinary skill by routine methods known in the art.

Moreover, a “treatment” or “prevention” regime of a subject with a therapeutically effective amount of an agent may consist of a single administration, or alternatively comprise a series of applications. For example, the agent may be administered at least once a week. However, in another embodiment, the agent may be administered to the subject from about one time per week to about once daily for a given treatment. The length of the treatment period depends on a variety of factors, such as the severity of the disease, the age of the subject, the concentration and the activity of the agent, or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required.

The term “subject” or “animal” as used herein includes all members of the animal kingdom including mammals, suitably humans, birds, aquatic species and ruminants.

The term “administering” is defined as any conventional route for administering an agent to a subject for use, for example, in treating a parasitic or inflammatory condition, disease or disorder, as is known to one skilled in the art. This may include, for example, administration via the parenteral (i.e. subcutaneous, intradermal, intramuscular, etc.) or mucosal surface route. In other embodiments this may include oral administration or topical administration. The dose of the agent may vary according to factors such as the health, age, weight and sex of the animal. The dosage regime may be adjusted to provide the optimum dose. One skilled in the art will appreciate that the dosage regime can be determined and/or optimized without undue experimentation.

The term “pharmaceutically acceptable” means compatible with the treatment of animals, suitably humans, birds and ruminants.

Administering a composition to a cell includes in vivo, ex vivo and in vitro treatment.

Accordingly, the present disclosure provides a process of obtaining a flax extract comprising subjecting seeds to a water extraction process followed by a mechanical extraction process and purifying the liquid therefrom.

In one embodiment, the process comprises:

a) washing flax seeds;

b) subjecting the washed flax seeds to the water extraction process;

c) removing the water soluble fraction;

d) subjecting the extracted seeds of c) to the mechanical extraction process; and

e) purifying the liquid from d).

The objective of the washing step is to remove impurities, such as dirt and other contaminants, from the seed material prior to further processing. In an embodiment, the flax seeds are washed in water at a temperature of 20 to 70 degrees Celsius. In another embodiment, the seeds are washed at a ratio of 1:10 to 1:2 seed weight:water volume. In the washing process, the seeds may be soaked in the water for several minutes to one hour. The washed seeds are optionally strained and rinsed under running water. The washing may be repeated as needed.

As used herein, the term “soaked” means that the seeds are submerged in water.

The term “water extraction” as used herein refers to a process of exposing the seeds to water, optionally hot water, to achieve the removal of water soluble compounds. The water extraction is optionally performed under pressure, for example pressure generated by a pressure cooker, for fast extraction. Alternatively, a similar effect can be obtained at a lower temperature under agitation for a longer period of time.

The objective of extraction includes, without limitation, 1) separating soluble components of the material from insoluble components; 2) isolating a desirable fraction; and 3) facilitating exposure of bioactive compounds to improve bioavailability.

In an embodiment, the water extraction is carried out at a temperature of 20 to 120° C., optionally, at about 110-120° C. The extraction is optionally carried out at a ratio of 1:100 to 1:4 seeds to water.

A person skilled in the art would understand that if the water extraction process is carried out at the lower end of the temperature range that the extraction process will require further time, for example, at least 4 to 8 hours to allow for removal of the water soluble compounds. For water extraction at the higher end of the temperature range, the extraction process is carried out for 1 to 4 hours.

The water extraction process step may be repeated as required to remove the water soluble compounds. The water-extracted seeds are optionally strained and rinsed under running water.

In an embodiment, the water extraction process is repeated at least once prior to purifying the liquid in e).

The term “mechanical extraction” as used herein refers to the process of disrupting the seed structure and exposing the biologically active compounds.

In one embodiment, the mechanical extraction process comprises mechanical shear force disruption, microfluidization, or ultrasonic-assisted extraction or a combination thereof. The mechanical extraction process is optionally performed at a ratio of 1:100 to 1:20 seeds to water.

The phrase “mechanical shear force disruption” as used herein refers to the mechanical disruption, for example, via a blender, homogenizer or grinder.

The term “microfluidization” as used herein refers to a process of physical disruption via two high pressure streams of liquid (water or water based) at high velocities.

The term “ultrasonic-assisted extraction” as used herein refers to physical disruption via a sonicator using ultrasound waves. A person skilled in the art could use any ultrasonic processor. For example, in the Examples described herein, the seeds were extracted using an ultrasonic processor UIP 1000hd (Hielscher Ultrasonics GmbH, Germany) fitted with a BS2d34 sonotrode and B2-1.2 booster.

In an embodiment, the ultrasonic-assisted extraction is carried out at a frequency of 20 kHz and the treatment parameters are an amplitude of 100%, a power output of 60-70 W/cm² for 15 minutes at 70° C. A person skilled in the art would understand that a similar effect can be obtained using a different instrument with different frequency, amplitude or power output at different (longer or shorter) time periods for processing as well as different temperatures.

In yet another embodiment, the seeds are washed after the water extraction process and before the mechanical extraction process, optionally under running water.

In a further embodiment, the liquid is purified by subjecting the seeds to centrifugation, gravity sedimentation and/or filtration after the mechanical extraction process and collecting the liquid therefrom.

In a particular embodiment, the process comprises:

a) washing flax seeds;

b) subjecting the washed flax seeds to a subcritical water extraction process;

c) removing the water soluble fraction;

d) subjecting the extracted seeds of c) to mechanical shear force grinding;

e) subjecting the ground seeds of d) to the ultrasonic-assisted extraction; and

f) purifying the liquid from e).

In another aspect, the present disclosure provides a flax extract produced by a process disclosed herein. In an embodiment, the flax extract produced by a process disclosed herein has anti-parasitic and/or anti-inflammatory activity.

In another aspect, the present disclosure provides a feed or food stuff, pharmaceutical composition or a food supplement composition, comprising the flax extract produced by a process disclosed herein. In another embodiment, the composition is in the form of a liquid, paste, gel or solid.

In yet another aspect, the present disclosure provides methods and uses of the flax extract, feed or food stuff, pharmaceutical composition or food supplement for treatment.

In one embodiment, there is provided a method of treating a parasitic infection comprising administering the flax extract, feed or food stuff, pharmaceutical composition or a food supplement composition disclosed herein to an animal in need thereof. Also provided herein is use of the flax extract, feed or food stuff, pharmaceutical composition or a food supplement composition disclosed herein for treating a parasitic infection in an animal in need thereof. Further provided is the flax extract, feed or food stuff, pharmaceutical composition or a food supplement composition disclosed herein for use in treating a parasitic infection in an animal in need thereof. Even further provided is use of a flax extract produced by a process disclosed herein for the manufacture of a medicament for treating a parasitic infection in an animal in need thereof.

The term “parasitic infection” as used herein refers to an infection caused by any parasite that has an oocyst developmental stage.

In one embodiment, the parasitic infection is a coccidial infection. In another embodiment, the parasitic infection is a Capillaria infection. In yet another embodiment, the parasitic infection is a trichomoniasis infection.

In another embodiment, there is provided a method of treating an inflammatory condition, disease or disorder comprising administering the flax extract, feed or food stuff, pharmaceutical composition or a food supplement composition disclosed herein to an animal in need thereof. Also provided herein is use of the flax extract, feed or food stuff, pharmaceutical composition or a food supplement composition disclosed herein for treating an inflammatory condition, disease or disorder in an animal in need thereof. Further provided is the flax extract, feed or food stuff, pharmaceutical composition or a food supplement composition disclosed herein for use in treating an inflammatory condition, disease or disorder in an animal in need thereof. Even further provided is use of a flax extract produced by a process disclosed herein for the manufacture of a medicament for treating an inflammatory condition, disease or disorder in an animal in need thereof.

In an embodiment, the inflammatory condition, disease or disorder affects the integument, mucosal membranes, intestine, or stomach of the animal.

In another embodiment, the inflammatory condition, disease or disorder is an inflammatory bowel lesion, necrotic enteritis, gizzard ulceration or skin lesion (e.g. foot pad necrosis).

In an embodiment, the animal is an agricultural animal, a zoo animal, a domestic animal, aquatic species or a companion animal. In one embodiment, the animal is a bird. In another embodiment, the animal is a human. In yet another embodiment, the animal is a ruminant. In yet a further embodiment, the aquatic species is a fish or shellfish, such as a shrimp.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Preparation of Flax Extract

Flax seeds were washed to remove impurities and contaminants, such as dirt, by submerging the seeds in hot water (approximately 70° C.) and soaking the seeds for several minutes or up to 1 hour. The temperature of the water is not critical and the temperature of the water could range from room temperature (about 21° C.) to about 70° C. The seeds were then strained through a colander, and rinsed 3 times under running water. In this process, “soaked” means that the seeds were totally submerged in water. In this example, 500 g of seeds were soaked in 2 L of tap water (1:4 w/v ratio). The process of washing and rinsing was repeated as needed until the seeds appeared to be clean.

The washed seeds were then subjected to a sub-critical water extraction process at a temperature of approximately 110° C. to 120° C. for 4 hours. In this example, 500 g of seeds processed in the first step (soaking) were extracted with 4 L of water (a seeds to water ratio of 1:8 w/v). In this example, the step of sub-critical water extraction was repeated two times. Upon completion of the sub-critical water extraction, the extracted seeds were strained through a colander and rinsed several times under running water.

Following the sub-critical water extraction step, the water-soluble fraction was discarded and the washed seeds were then subjected to mechanical shear force grinding, followed by ultrasonic-assisted extraction.

The mechanical disruption of seeds was performed in water using a ratio of seeds to water of 1:20 w/v (100 g of the seeds were homogenized in 2 L of water). After completion of homogenization, the obtained material was further processed using ultrasonic-assisted extraction.

Ultrasonic-assisted extraction was performed in water at a ratio of 100 g of the washed seeds per 2 L water (1:20 w/v) using an ultrasonic processor UIP 1000hd (Hielscher Ultrasonics GmbH, Germany) fitted with a BS2d34 sonotrode and B2-1.2 booster. The processor was operated at a frequency of 20 kHz. Ultrasonic treatment parameters were: amplitude 100%, power output 60 to 70 W/cm², 15 minute treatment time, and a temperature of 70° C. Each ultrasonic extraction was performed twice. After ultrasonic extraction, the preparation was centrifuged at approximately 6,000 g for 15 min to remove the extracted meal and the supernatant was collected and filtered using a 0.45 μm membrane. The filtered supernatant is the simplified flax extract.

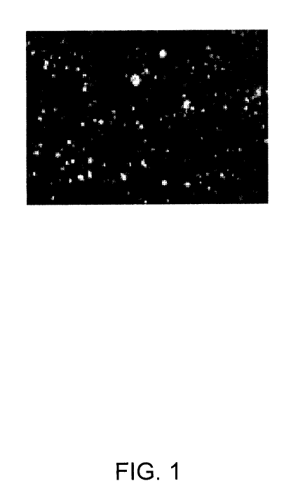

The simplified flax extract is a water-based extract with a pH of 5.4 to 5.7. On visual evaluation, it has the appearance of milky fluid, with creamy off-white/greyish coloration. On light microscopic examination, the flax extract appears to form water based emulsion consisting of micro and (mostly) nano-sized vesicles with well defined, globe shaped structures (FIG. 1).

### Example 2

### Effect of the Simplified Flax Extract on Eimeria Oocyst Sporulation In Vitro

Anticoccidial potency of the flax extract was evaluated using Eimeria oocysts isolated from field cases of clinical coccidiosis in chickens, pigeons, sheep, and cattle. Eimeria oocysts were dispensed into four test tubes, and the tubes were randomly assigned to either control (using water as the incubation medium) or treatment (using water amended with a defined content of the flax extract (e.g. 10, 5, or 2.5% v/v concentration)), said flax extract prepared as described in Example 1. Sporulation frequency was evaluated after approximately 16, 36, and 72 hours of incubation.

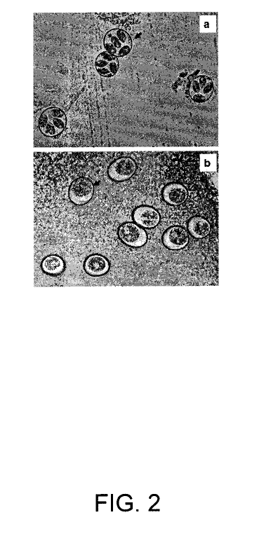

Oocysts in the control samples showed signs of vigorous sporulation, whereas sporulation in the treated samples was reduced. A representative micrograph illustrating this difference is presented in FIG. 2. Overall, the trial showed that following 72 hours of incubation, 82-98% of Eimeria oocysts had completed sporulation in the control samples. In contrast, the flax extract showed a strong inhibitory effect. In the samples treated with the flax extract, approximately 92-98% of oocysts failed to sporulate within 72 hours. The results from one representative experiment are summarized below:

| | 16 hours | | 36 hours | | 72 hours | |
|---|---|---|---|---|---|---|
| | | Not | | Not | | Not |
| | Sporu- | Sporu- | Sporu- | Sporu- | Sporu- | Sporu- |
| | lated | lated | lated | lated | lated | lated |
| Control | 36 | 64 | 57 | 43 | 82 | 18 |
| Treatment | 1 | 99 | 3 | 97 | 8 | 92 |

It was also noted that among the oocysts that did manage to sporulate despite treatment, the development of spores did not progress to sporozoites. Thus, without wishing to be bound by theory, it appears that this fraction is effective in stopping the development of infectious forms of coccidia. These effects on Eimeria oocysts differ from those associated with cyclolinopeptides which can be characterised as oocyte injury resulting in blebbing (WO2013/091071).

### Example 3

### Effects of Simplified Flax Extracts on Eimeria Cycle In Vivo Using a Chicken Model

Trial 1, Pen Study:

A group of 25 broilers was raised on straw bedding until the age of three weeks. Periodic sampling of litter showed no presence of Eimeria oocysts. At the age of three weeks, all birds were challenged with Eimeria oocysts. The challenge was designed to mimic natural infection, where fecal matter from a donor bird previously infected with coccidian oocysts was incubated in drinking water overnight. The water containing sporulated oocysts was added to the bell drinker for 3 days. A sample of manure taken one week after the start of the Eimeria challenge confirmed that the birds were shedding oocysts, so it was concluded that the challenge resulted in coccidial infection.

Ten (10) randomly selected birds from the infected pen were moved to another pen, which upon initial evaluation was deemed to be free of Eimeria oocysts. The birds in this pen were not treated, and served as controls for the pen study. Fresh straw bedding was placed in the infected pen, and birds remaining in the infected pen were treated with flax extract, prepared as described in Example 1, added to the drinking water at a rate of 5% v/v. After 4 days, samples of manure from both the treatment and control pens were examined for Eimeria oocysts. A manure sample from the control pen showed oocysts in fecal matter and the hemocytometer count was approximately 1×10⁵ oocysts/g of litter. There were no detectable oocysts in the litter sample from the pen housing initially infected birds treated with flax extract as assessed by hemocytometer (quantitative detection limit approximately 1000 oocysts/g fecal matter), but a small number of oocysts was apparent on flotation test (estimated <1000 oocysts/g fecal matter).

Trial 2, Individual Subjects Study:

A group of 20 chickens were directly challenged with Eimeria at the age of 14 days. Nine days later, one of the birds showed signs of illness (somnolence, ruffled feather). It was noted that droppings from these birds were stained with streaks of blood. Fecal matter was evaluated for Eimeria, and showed numerous oocysts on flotation. Oocyst count in this sample using a hemocytometer showed 3×10⁶ oocysts/g of fecal matter. This chicken was moved to a cage with a raised metal floor, and was offered drinking water amended with 10% v/v of flax extract, prepared as described in Example 1. Following approximately 20 hours, fecal matter was examined. The fecal matter contained very little blood streaks and the number of fecal oocysts was considerably reduced, with an estimated oocyst count showing <1000 oocysts/g. Oocysts from this bird were harvested prior to and after treatment, and subjected to a routine sporulation test. The sporulation rate of the oocysts prior to treatment was approximately 99%, whereas sporulation of oocysts harvested following treatment with flax extract was approximately 65%. In addition, many oocysts that managed to sporulate appeared sickly. Further observations suggest that the majority of sporulated oocysts would fail to develop sporozoites. No further evaluation was performed because this bird died of unrelated causes.

Twelve days after challenge, another bird showed clinical signs (somnolence, ruffled feather), and droppings stained with streaks of blood. Fecal material was examined for oocysts, and showed numerous oocysts on flotation test. The bird was placed in a cage and provided drinking water amended with 10% v/v flax extract. The sporulation rate of the oocysts harvested prior to treatment was approximately 99%, whereas sporulation of oocysts harvested 24 hours following treatment with flax extract was approximately 80%. Many oocysts that managed to sporulate appeared sickly. There were no detectable oocysts in fecal samples from this bird examined at 48 hours and 72 hours following treatment.

Seventeen days after challenge, 3 birds were examined for fecal content of oocysts, and showed numerous oocysts on flotation. An oocyst count using a hemocytometer showed approximately 5×10⁴ oocysts/g for birds #1 and 2, and 3×10⁶ oocysts/g for bird #3. The birds were placed in a cage with a raised metal floor, and treated with 10% v/v flax extract added to drinking water. The following day, fecal matter was examined. An oocyst count using a hemocytometer showed practically no detectable Eimeria oocysts in droppings from bird #1. The oocyst count in bird #2 showed numbers <1000/g, and in bird #3 5.6×10⁴ oocysts/g. Two days later, there were no detectable oocysts in fecal samples from these birds. The sporulation rate of the oocysts isolated from these chickens prior to treatment was approximately 95 to 99%. Sporulation of oocysts harvested approximately 24 hours following treatment with flax extract was approximately 66% for bird #3 and 37% for bird #2.

Trial 3:

During the 4^{th} week of age, 12 broilers were challenged with mild doses of sporulated Eimeria oocysts administered in drinking water. On day 3, droppings from all challenged birds were evaluated for oocysts, and all samples confirmed that the birds were shedding small numbers (˜1000 oocysts per g of fecal matter). The challenged birds were randomly divided into two groups (six birds per group) and placed in separate pens. On day 6 post challenge, droppings from all challenged birds were evaluated again for oocysts content, and this time all birds showed significant numbers of oocysts in droppings with average of approximately 102,000 oocysts/g fecal matter in group 1, and 120,000 oocysts/g fecal matter in group 2. The birds in group 2 were assigned to treatment group and offered drinking water amended with flax extract (10%), whereas the birds in group 1 served as controls. All chickens were monitored daily for any signs of disease.

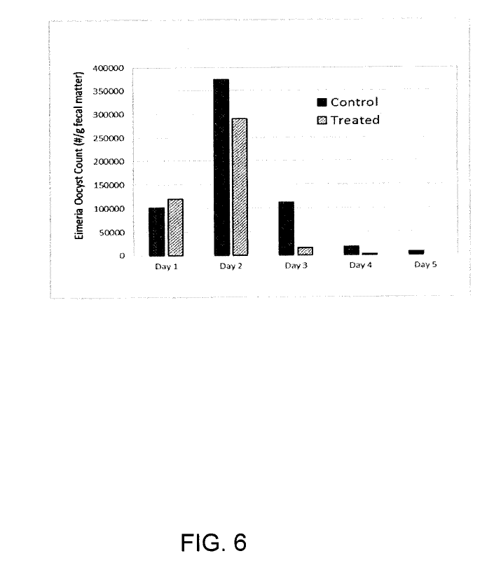

Oocysts counts were performed in droppings from each bird in both groups daily, for 5 consecutive days. For fecal matter collection, each bird was placed in individual cages lined with paper towels, and excreta were collected for 4 to 6 hours. Following droppings collection, the birds were returned to respective treatment or control pens. The excreta from each cage was transferred to plastic containers, and net weight of the collected droppings was measured. Next, the fecal samples were homogenized with reverse osmosis (RO) water at a ratio 1:5 w/v, and strained through cheesecloth. Oocyst count was performed in a hemocytometer. The results from this trial are summarised in FIG. 6.

Of note, the numbers of oocysts in fecal matter of challenged birds in both groups peaked on day 7 post challenge (day 2 of treatment), and then gradually declined over the next 3 days. This pattern of oocyst shedding in feces is typical. However it is noteworthy that oocyst counts in control birds were much higher in comparison to broilers drinking water amended with flax extract (FIG. 6). On the second day, the number of oocysts was approximately 20 to 30% lower in the treated birds. On the 3^{rd} day of treatment, the oocyst count in treated birds declined approximately 8 to 10 fold relative to controls, and by the 5^{th} day on treatment the number of oocysts in feces was below measurable detection limit.

Based on responses of chickens challenged with coccidia, the treatment with flax extract was effective in reducing oocyst shedding. Infected chickens drinking water amended with flax extract showed lower oocyst shedding in the excreta within 24 hours following treatment, and cleared oocysts shedding at least 2 days earlier in comparison to the infected controls. Oocysts shed in the feces increase the risk of wide-spread coccidial infection in the flock and therefore this observation indicates that administration of flax extract will help to control coccidiosis.

### Example 4

### Response of Commercial Broiler Chickens to Flax Extract

To assess the general response of commercial boiler chickens to flax extract, two trials were carried out. The first trial used one day old commercial broilers raised as per standard industry practice. At the age of 14 days, the birds were randomly divided into two experimental groups. Group 1 (n=25) received the flax extract added to drinking water at a concentration of 5% (v/v) and group 2 (n=25) was a control group that received normal drinking water. All chickens were offered standard commercial broiler starter diet ad libitum. The responses of chickens were monitored for 21 days.

The second trial was focused on evaluation of responses in commercial broilers at increased risk of morbidity/mortality. 40 broilers (one day old) were randomly allocated to two experimental groups. Group 1 (n=20) received the flax extract added to drinking water a rate of 5% (v/v), group 2 (n=20) was a control group offered normal drinking water. The chickens were exposed to the respective treatments for 21 days.

Standard experimental protocol included daily monitoring of morbidities and mortalities. The birds were weighed at weekly intervals. At the end of the observation period, all birds were euthanized and subjected to detailed post mortem examination.

### Trial 1: Normal Population of Broilers

When evaluating any candidate health product for administration in water, one major concern is water palatability. This is important because if treated water is refused by the animals, then its scope of application is reduced, or even rendered impractical for preventative application.

Over the 21 days of trial 1, there was no evidence of any adverse effects associated with the administration of flax extract in water. In addition, the amendment of drinking water with flax extract had no effect on water consumption and feed intake by the broilers. Animals offered drinking water amended with the flax extract showed a slightly better growth rate and had fewer incidents of enteritis and foot pad lesions compared to controls.

| | Average Body | | |
|---|---|---|---|
| | Weight (grams) | | |
| | Day 7 | Day 14 | Day 21 |
| Control Group | 141.6 | 497.9 | 950.9 |
| Treatment Group | 162.1 | 523.9 | 981.8 |

There were no mortalities during the course of trial 1, and all birds appeared physiologically normal throughout. The trial was terminated during the fourth week and all surviving birds from control and treatment groups were subjected to clinical evaluation, followed by euthanasia, in order to evaluate the effects of the treatments on internal organs.

On clinical examination prior to euthanasia, all birds from both control and treatment groups appeared normal, but some birds in both control and treatment groups were considerably smaller.

Gross pathological changes in the liver, heart, and bones were observed in some birds from both groups, but overall pathology was unremarkable as would be typically expected in broilers on cross-sectional evaluation. So, it was concluded that administration of the flax extract did not cause any specific gross patho-physiological changes.

However, there was a noticeable difference between the control and treatment groups in the incidence and severity of some common pathologies. In particular, among the birds that were growing at a slower rate some interesting patterns were observed. Specifically, it was noted that “ill-thrift birds—those that failed to maintain the expected growth rate in the absence of recognizable disease—had a high incidence of generalized subclinical enteritis.

Subclinical enteritis represents pathology of inflammatory responses in intestinal mucosa characterized on gross examination by focal, multifocal, or locally extensive hyperemia. This process involves infiltration of the villi lamina propria with cellular elements, and the production of numerous chemical mediators that aggravate the inflammatory process.

Subclinical enteritis is seen commonly in otherwise normal commercial broilers, with variable frequency of occurrence typically ranging from 10 to 30% in most situations, but incidences as high as 40 to 50% were observed in our studies.

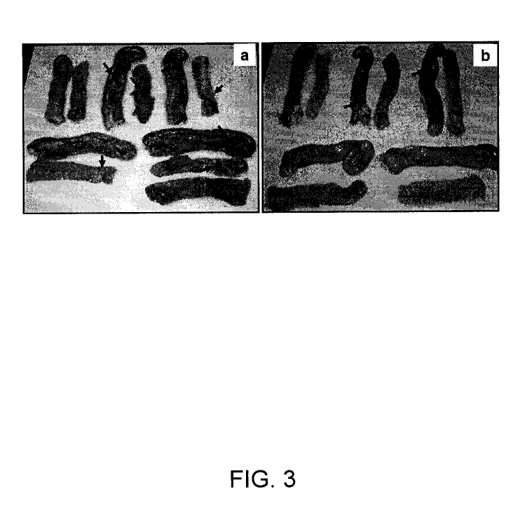

In trial #1, out of 25 chickens in the control group, 5 birds (20%) showed moderate to severe gross lesions in the mucosa of duodenum, jejunum, and ileum. Interestingly, in the group of birds drinking water amended with the flax extract, the incidence of enteritis lesions was reduced, with only 3 out of 25 (12%) chickens showing inflammatory lesions in the intestinal mucosa (FIGS. 3a and 3b).

Notably, the inflammatory responses in the intestinal mucosa are considerably pronounced with respect to both magnitude and severity in control broilers (FIG. 3a, arrows) in comparison to broiler drinking water amended with flax extract fraction B (FIG. 3b, arrows). This indicates that the flax extract may contain biologically active compounds capable of controlling the pathological course of the inflammatory process.

### Trial 2: Population of Broilers at Risk of Health Problems

The birds used during trial 2 were at a higher risk of health problems, and morbidity and mortality were observed in both control and treatment groups. However, the incidences of morbidity and mortality were considerably lower in the treatment group in comparison to the control group, as illustrated below:

| | Morbidity and Mortality |
|---|---|
| Control Group | 5/20 |
| | (25%) |
| Flax extract treatment | 3/20 |
| | (15%) |

Gross post mortem examination revealed that morbidities and mortalities during the course of the experiment were associated with common health problems routinely seen in commercial broiler flocks including pericarditis, polyserositis, air saculitis, and enteritis.

The trial was terminated during the fourth week and all surviving birds from control and treatment groups were subjected to clinical evaluation, followed by euthanasia in order to evaluate the effects of the treatments on internal organs.

The anti-inflammatory effect of the flax extract that was observed in trial 1 was confirmed in trial 2. However, in trial 2 necrotic enteritis was observed in addition to generalized enteritis.

Gross post mortem evaluation revealed necrotic lesions in the intestinal mucosa in specimens from both control and treatment groups, but the incidence and severity of the lesions were considerably lower in the chickens drinking water amended with the flax extract, as illustrated below:

| | Cases of Necrotic Enteritis/ |
|---|---|
| | Number of Sampled (%) |
| Control Group | 7/15 (severe lesions) |
| | (46.7%) |
| Flax extract | 2/17 (mild lesions) |
| treatment | (11.8%) |

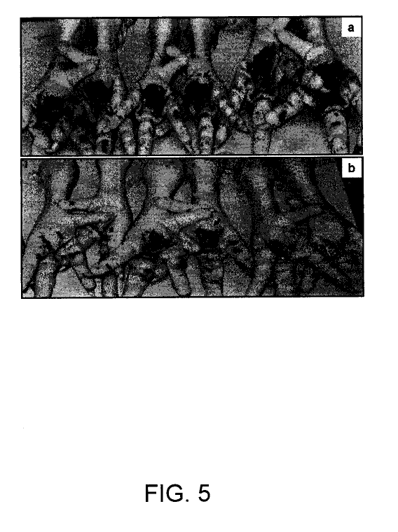

Birds in the control group were 2.64 times more likely to develop necrotic enteritis than those in the treated group. It is also noteworthy that the risk of occurrence of the necrotic lesions was considerably reduced (relative risk reduction 62.2%) in the treatment group in comparison to the control group. Interestingly, in addition to lower incidence, the severity of lesions was considerably lower in the broilers drinking water amended with flax extract (FIG. 5).

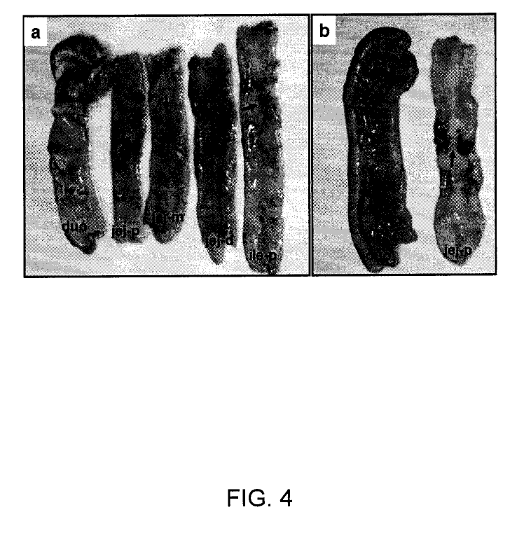

As noted above, the necrotic changes in the intestinal mucosa are considerably more pronounced with respect to both magnitude and severity in control broilers (FIG. 4a, arrows) in comparison to broilers drinking water amended with the flax extract (FIG. 4b, arrows). Typically, the necrotic lesions of the intestinal mucosa seen in the control birds were characterized as moderate to severe, multifocal or locally extensive, and were spread throughout the duodenum, jejunum, and ileum. On the other hand, the lesions observed in chickens drinking water amended with the flax extract appeared mild to moderate, and were mostly confined to the duodenum and proximal jejunum.

In addition to beneficial effects on general health, and the severity of enteritis specifically, the flax extract treatment also affected the development of foot pad lesions. These lesions commonly occur in fast growing broilers in a commercial setting and are characterized by progressive necrotic erosions of the soft tissue.

In a commercial situation, the incidence of foot pad lesions can be observed in some 20 to 80% of otherwise normal broiler populations. Any foot pad lesions are undoubtedly a cause of distress, but in more severe cases these lesions are likely associated with severe pain and markedly decreased walking ability.

During the course of the trials it was observed that, in comparison to control broilers drinking untreated water, the broilers offered drinking water amended with the flax extract showed considerably lower incidence of foot pad lesions (FIG. 5).

Taken together, the trials using the avian model demonstrated that flax extract has the potential to produce several positive health outcomes. The analysis of these findings is summarized below.

Summary of Results from Trials 1 and 2

Overall analysis of positive outcomes associated with flax extract administration in a normal population of broiler chickens and in a population at risk:

| | Normal Population | | | Population at Risk | | |
|---|---|---|---|---|---|---|
| | | | Odds | | | Odds |
| | Control | Treated | Ratio | Control | Treated | Ratio |
| Outcome | (N = 25) | (N = 25) | (95% CI) | (N = 20) | (N = 20) | (95% CI) |
| Mortality | 0 | 0 | NA | 5 | 3 | 1.9 |
| | | | | | | (0.4-8.6) |
| Inflam- | 5 | 3 | 1.8 | 3 | 1 | 3.4 |
| matory | | | (0.4-7.9) | | | (0.4-25.8) |
| Bowel | | | | | | |
| Lesions | | | | | | |
| Necrotic | 0 | 0 | NA | 8 | 2 | 6.0 |
| Enteritis | | | | | | (1.2-29.6) |
| Severe Foot | 15 | 5 | 6.0 | 12 | 6 | 3.5 |
| Lesions | | | (1.6-20.6) | | | (1.0-12.7) |

*Odds Ratio and Confidence Intervals (CI) are based on Farrington & Manning Score.

### Additional Observations of Health Effects in Chickens.

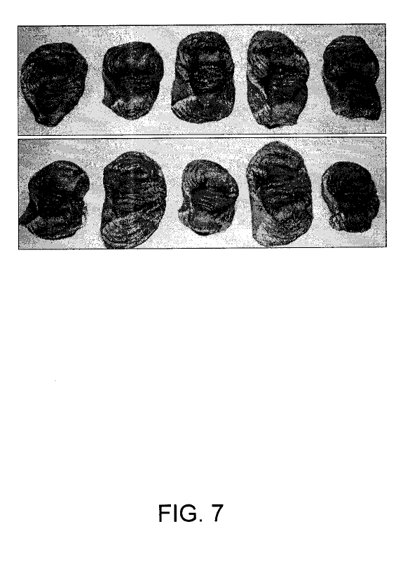

Broilers drinking water amended with the flax extract showed a notable reduction in lesions (erosions and ulceration) of the chicken stomach (gizzard). On gross post-mortem examination these appeared as deep ulceration and erosion of the stomach mucosal lining (FIG. 7, from Trial 3 but representative of overall observations).

Two sets of specimens shown in both panels without arrows represent relatively normal gizzard mucosa. Notably, the ulcerative lesions in controls are mostly multifocal and locally extensive, and are more pronounced with respect to frequency of occurrence (3 out of 5 specimens showed moderate to severe lesions), as well magnitude and severity. In contrast, lesions in broilers drinking water amended with flax extract were mostly focal, and occurred at lower frequency (2 out of 5 specimens showed mild to moderate lesions).

Gizzard ulceration is a common condition in commercial meat birds, such as chickens and turkeys (for review see Gjevre et al., 2013). The etiology of these lesions is not clear. However, the fact that these lesions can be alleviated by treatment with flax extract indicates potent anti-inflammatory effects associated with some bioactive compounds present in this extract.

### Example 5

### Observational Study of Treatment of Trichomoniasis in Pigeons

Trichomoniasis is a protozoan disease causes by Trichomonas gallinae, which is found in the mouth, throat, gastro-intestinal tract and upper respiratory tract of the affected birds. This condition is commonly seen in pet birds, garden birds, and many wild bird species. In most cases, if left untreated, the disease is fatal. The drugs of choice include antiprotozoal agents such as furasolidone and metronidazole.

Trichomoniasis is frequently a devastating disease in domestic pigeons, with very high morbidity and mortality rates. Five pigeons that showed advanced signs of trichomoniasis, as well as several others that showed mild signs were investigated. The common signs associated with trichomoniasis include decreased appetite, depression, somnolence, ruffled feathers, hunched back, and diarrhea. In order to treat these birds, simplified flax extract was blended with drinking water (5% v/v). The improvement in clinical status was apparent in a few days. In the end, all mildly affected pigeons recovered completely. Interestingly, however, 4 out 5 severely affected birds also fully recovered from the disease. Typically, mortality in pigeons showing severe signs of trichomoniasis approaches 100%. In light of this, the above described approach to use flax extract to treat trichomoniasis in birds appears to be very effective.

### Example 6

### Effects of Simplified Flax Extract on Parasitic Infections: Observations from Pigeons

This experiment was focused on evaluation of responses of Capillaria to flax extract treatment. Two consecutive trials were conducted at 4 week intervals using birds from the same flock. The focus was on birds showing signs of impending parasitic disease (somnolence, wet dropping, ill thrift). The birds suspected of developing parasitic disease were isolated from the flock and placed in cages with a raised wire floor.

In each trial, a total of 6 birds were examined. Droppings from individual birds were evaluated, and the results revealed that all birds were shedding Capillaria Columbae oocysts with counts ranging between 1000 and 5000 oocysts/g fecal matter. In each trial, three randomly selected birds were assigned to treatment group and offered drinking water amended with flax extract (10%) and the remaining 3 were used as controls.

After 7 days, droppings from each bird were evaluated for Capillaria. Oocysts from both control and treated birds were harvested, and incubated in vitro in water at room temperature (about 21° C.). The development of oocysts was evaluated at 24, 48, and 72 hours.

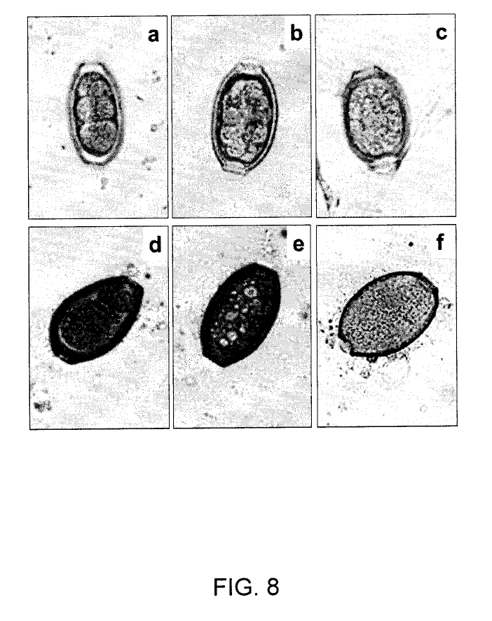

The results revealed that the numbers of Capillaria oocysts in fecal matter of treated birds declined by approximately 30 to 60% in comparison to controls. Consistently, it was also noted that the embryonation stages in vitro differed between oocysts from control birds as compared to treated birds. Evaluation of oocysts incubated in vitro showed clear differences in morphology (FIG. 8).

Approximately 70 to 80% of Capillaria oocysts isolated from control birds incubated in vitro showed signs of at least four-cell stage development at 48 hours (FIG. 8a), and at 72 hours, embryonation was advanced to at least the morula stage (FIG. 8b) or the early larva stage in the vast majority of oocysts (FIG. 8c). In contrast, approximately 50 to 70% of Capillaria oocysts isolated from birds treated with flax extract either failed to commence the embryonation process, or the oocysts' embryonation was halted at the two-cell stage (FIG. 8d). In addition, many oocysts isolated from treated birds that commenced embryonation appeared sickly, showing signs of rarified cellular content (FIG. 8d) and many eggs showed advanced pathological change such as cellular vacuolization (FIG. 8e). Some oocysts showed gross degradation of cellular material into an amorphous mass (FIG. 8f).

Based on the observations from this pigeon study, it appears that treatment with flax extract may be used to control capillariasis, and perhaps other parasitic infections associated with the genus of nematodes.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Calder, P. C. 2010. Omega-3 Fatty Acids and Inflammatory Processes. Nutrients 2010: 2, 355-374.
Gjevre, A. G., Kaldhusdal, M. and Eriksen, G. S. 2013. Gizzard erosion and ulceration syndrome in chickens and turkeys: a review of causal or predisposing factors. Avian Pathology 42: 207-303.
Gorski, A., Kasprzycka, M., Nowaczyk, M., Wieczoreck, Z., Siemion, I. Z., Szelejewski, W. and Kutner, A. 2001. Cyclolinopeptide: A novel immunosuppressive agent with potential anti-lipemic activity. Transplantation Proceedings 33: 553-553.
Huwiler, A. and Pfeilschifter, J. 2009. Lipids as targets for novel anti-inflammatory therapies. Pharmacol. Ther. 124: 96-112.
Matsumoto, T., Shishido, A., Morita, H., Itokawa, H. and Takeya, K. 2001. Cyclolinopeptides F-I, cyclic peptides from linseed. Phytochemistry 57: 251-260.
Morita, H., Shishido, A., Matsumoto, T., Itokawa, H., Takeya, K., Itokawa, H., Hirano, T. and Oka, K. 1997. A new immunosuppressive cyclic nonapeptide, cyclolinopeptide B from Linum usitatissimum. Bioorganic and Medicinal Chemistry Letters: 7, 1269-1272.
Peek, H. W and Landman, W. J. M. 2011. Coccidiosis in poultry: anticoccidial products, vaccines and other prevention strategies. Veterinary Quarterly 31: 143-161.
Picur, B., Cebrat, M., Zabrocki, J. and Siemion, I. Z. 2006. Cyclopeptides of Linum usitatissimum. J. Pept. Sci. 12: 569-574.
Russo, G. L. 2009. Dietary n-6 and n-3 polyunsaturated fatty acids: from biochemistry to clinical implications in cardiovascular prevention. Biochem. Pharmacol. 77: 937-946.
Siemion, I. Z., Cebrat, M. and Wieczorek, Z. 1999. Cyclolinopeptides and their analogs—a new family of peptide immunosuppressants affecting the calcineurin system. Arch. Immunol. Ther. Exp. 47: 143-153.
Wieczorek, Z., Bengtsson, B., Trojnar J. and Siemion, I. Z. 1991. Immunosuppressive activity of cyclolinopeptide A. Peptide Research 4: 275-283.

1. A process of obtaining a flax extract having anti-parasitic and/or anti-inflammatory activity comprising subjecting flax seeds to a water extraction process followed by a mechanical extraction process and purifying the liquid therefrom.

2. The process of claim 1 comprising:
a) washing flax seeds;
b) subjecting the washed flax seeds to the water extraction process;
c) removing the water soluble fraction;
d) subjecting the extracted seeds of c) to the mechanical extraction process; and
e) purifying the liquid from d).

3. The process of claim 2, wherein the flax seeds are washed in (a) in water at a temperature of 20 to 70° C.

4. The process of claim 3, wherein the seeds are washed at a ratio of 1:10 to 1:2 w/v seeds:water.

5. (canceled)

6. (canceled)

7. The process of claim 1, wherein the water extraction is carried out at a temperature of 20 to 120° C.

8. The process of claim 7, wherein the water extraction process is carried out at about 110-120° C.

9. The process of claim 1, wherein the water extraction process is carried out at a ratio of 1:100 to 1:4 w/v seeds to water.

10. The process of claim 1, wherein the water extraction process is repeated multiple times before the mechanical extraction process.

11. (canceled)

12. The process of claim 1, wherein the mechanical extraction process comprises mechanical shear force mixing, homogenization, microfluidization, physical grinding, ultrasonic-assisted extraction or a combination thereof.

13. The process of claim 1, wherein the mechanical extraction process is performed at a ratio of 1:100 to 1:20 w/v seeds to water.

14. (canceled)

15. The process of claim 1, wherein the water extraction process is repeated at least once after the mechanical extraction process prior to purifying the liquid in e).

16. The process of claim 1, wherein the liquid is purified by subjecting the seeds to centrifugation, gravity sedimentation and/or filtration and collecting the liquid therefrom.

17. The process of claim 1 comprising:
a) washing flax seeds;
b) subjecting the washed flax seeds to a subcritical water extraction process;
c) removing the water soluble fraction;
d) subjecting the extracted seeds of c) to mechanical shear force grinding;
e) subjecting the ground seeds of d) to the ultrasonic-assisted extraction; and
f) purifying the liquid from e).

18. A feed or food stuff, pharmaceutical composition or a food supplement composition, comprising the flax extract produced by the process of claim 1.

19. (canceled)

20. A method for treating a parasitic infection in an animal in need thereof comprising administering the feed or food stuff, pharmaceutical composition or a food supplement composition of claim 18 to the animal in need thereof.

21. The method of claim 20, wherein the parasitic infection is a coccidial infection, a trichomoniasis infection or a capillarial infection.

22. (canceled)

23. (canceled)

24. A method of treating a inflammatory condition, disease or disorder in an animal in need thereof comprising administering the feed or food stuff, pharmaceutical composition or a food supplement composition of claim 18 to the animal in need thereof.

25. The method of claim 24, wherein the inflammatory condition, disease or disorder affects the integument, stomach or intestine of the animal.

26. The method of claim 24, wherein the inflammatory condition, disease or disorder is an inflammatory bowel lesion, necrotic enteritis, or skin lesions.

27. The method of claim 20, wherein the animal is an agricultural animal, a zoo animal, a domestic animal or a companion animal.

28.-31. (canceled)
